(19) United States
(12) Patent Application Publication
(10) Pub. No.: US 2024/0145059 A1
(43) Pub. Date: May 2, 2024

(54) METHOD AND SYSTEM FOR DISCOVERING ADVERSE DRUG REACTION SIGNAL BASED ON CAUSAL DISCOVERY

(71) Applicant: ZHEJIANG LAB, Hangzhou (CN)

(72) Inventors: Jingsong LI, Hangzhou (CN); Yu WANG, Hangzhou (CN); Shuang MA, Hangzhou (CN); Yu TIAN, Hangzhou (CN); Tianshu ZHOU, Hangzhou (CN)

(21) Appl. No.: 18/364,470

(22) Filed: Aug. 2, 2023

(30) Foreign Application Priority Data
Nov. 2, 2022 (CN) 202211361950.8

(51) Int. Cl. G16H 20/10; G16H 10/20; G16H 10/60

(52) U.S. Cl. CPC G16H 20/10; G16H 10/20; G16H 10/60

(57) ABSTRACT

Disclosed is a method and a system for discovering adverse drug reaction signals based on causal discovery. According to the present application, a causality is introduced in the process of discovering adverse drug reaction signals by using electronic medical record data, the data dimension in real-world electronic medical record data is maximally reserved, a Bayesian network structure containing causal effects, as well as a set of confounding factors which plays a role in both a medication intervention and an occurrence of an adverse event are constructed. The method of constructing the set of confounding factors starts from the data, without artificial access and prior knowledge, and retains the confounding factors in the real world to the greatest extent. A medication intervention group and a control group are constructed based on these confounding factors, and the randomized controlled trial is simulated.

### CROSS-REFERENCE TO RELATED APPLICATIONS

The present application claims priority to Chinese Patent Application No. 202211361950.8, filed on Nov. 2, 2022, the content of which is incorporated herein by reference in its entirety.

### TECHNICAL FIELD

The present application belongs to the technical field of medical information, in particular to a method and a system for discovering adverse drug reaction signals based on causal discovery.

### BACKGROUND

Adverse drug reactions (ADRs) can be defined as “an appreciably harmful or unpleasant reaction resulting from an intervention related to the use of a medicinal product”. This definition includes reactions due to errors, misuse or abuse, suspicious reactions to drugs used without permission or off-label use, and reactions caused by the use of normal doses of drugs. Over the past half-century, the primary method for detecting potential ADRs has been through spontaneous reporting systems. These systems have been widely implemented worldwide and have proven to be highly effective in identifying rare and uncommon adverse events (occurring in less than 1% of treated patients) and those that are typical drug-induced symptoms. However, spontaneous reporting systems still suffer from underreporting, selective reporting, and duplicate reporting.

At present, China has basically established a monitoring system for adverse drug reactions. The invention for a patent with an authorization announcement number of CN104765947B and the invention for a patent with an authorization announcement number of CN111402971B both disclose the method of mining potential adverse drug reactions based on the spontaneous reporting of big data of adverse drug events. With the continuous development of medical informatization, more and more data are accumulated in medical information systems such as electronic medical records, which will bring new supplementary evidence for the discovery of adverse drug reactions based on spontaneous reporting system. According to the basic principles, ADR mining methods based on electronic medical record data can be divided into the following categories: methods based on ratio imbalance, traditional drug epidemiological design methods, symmetric analysis of prescription sequence, sequential statistical test, sequential association rules, supervised machine learning and tree scanning statistics. The invention for a patent “Intelligent Detection Method, Device, System and Computer Equipment for Adverse Drug Reactions” with an authorization announcement number of CN110322944B discloses a method for ADR discovery by using multi-source dynamic patient diagnosis and treatment data, which takes clear rules of adverse drug reactions as the reasoning basis and focuses on the judgment of adverse drug reactions for patients.

Clinical scenarios in the real world are more complicated than clinical trials. Doctors give drugs according to medical knowledge and experience. For example, they often give drugs individually according to patients' features, so the effects of drugs in the clinical process often show different features from those in clinical trials before marketing. Whether based on the data of a spontaneous ADR reporting system or electronic medical records, the existing ADR detection methods can be mainly divided into two categories: one is to make explicit reasoning and judgment based on the established knowledge of drugs and ADR; and the other one is based on data analysis or data mining. The former only applies the existing knowledge clinically, while the latter can only find the correlation between drugs and adverse reactions to a certain extent. Correlation does not mean that there is a causality, which will greatly reduce the possibility that the potential signals found will become new clinical evidence.

### SUMMARY

In view of the shortcomings of the prior art, it is an object of the present application to provide a method and a system for discovering adverse drug reaction signals based on causal discovery. According to the present application, causality is introduced in the process of discovering adverse drug reaction signals by using electronic medical record data, the data dimension in real-world electronic medical record data is retained to the maximum extent, a Bayesian network structure containing a causality is constructed, and a set of confounding factors which have effects on both medication intervention and adverse events is constructed, and a random controlled trial is simulated based on the set of confounding factors, so that the comparison of adverse drug reactions among groups has causal significance, and then an adverse drug reaction signals with the causality is generated.

The object of the present application is achieved through the following technical solutions.

According to a first aspect of this specification, a method for discovering adverse drug reaction signals based on causal discovery is provided and the method includes the following steps:
acquiring and cleaning real-world electronic medical record data;
selecting a target drug and an adverse event, marking use of the target drug as an index event and an appearance of a target adverse event as a marker event, and constructing a patient cohort according to a patient population in which the index event or the marker event occurs;
generating a set of confounding factors affecting both a medication intervention and an occurrence of an adverse reaction by constructing a Bayesian network containing a causal property; and
constructing cohorts of an intervention group and a control group based on the set of the confounding factors, simulating a randomized controlled trial, evaluating a difference in occurrences of adverse reactions between the intervention group and the control group, and generating an adverse drug reaction signal having the causality.

Further, the target drug is a single drug, or a type of drugs having a same efficacy, or a type of drugs having a same property.

The adverse event is defined by using a diagnosis, or a specific type of laboratory reports, or both the diagnosis and the specific type of laboratory reports.

Further, the patient population in which the index event or the marker event occurs is defined as an enrolled population, inclusion and exclusion criteria are defined to screen the enrolled population, the screened enrolled population constitutes the patient cohort, and the patient data in the patient cohort constitutes the enrolled patient dataset.

Further, a generation method of the set of the confounding factors is as follows:
marking patient data in the patient cohort as an enrolled patient dataset, containing a feature X_{index} indicating whether the index event occurs, a feature Xₘₐᵣₖₑᵣ indicating whether the marker event occurs, and other features of an enrolled patient extracted from the electronic medical record data;
forming a preliminary screened feature set by retaining features that will affect occurrence of the index event or the marker event through a single-factor logistic regression method; and
using the feature in the preliminary screened feature set as a node of the Bayesian network, learning a Bayesian network structure from the enrolled patient dataset according to a K2 algorithm, introducing a causality in a learning process of the Bayesian network structure, obtaining a parent node set of each node after a plurality of rounds of iterations, considering a common parent node of the features X_{index} and Xₘₐᵣₖₑᵣ as a factor playing a role in occurrence of both the index event and the marker event, and generating the set of the confounding factors.

Further, a node priority of the K2 algorithm is optimized, specifically: using a mutual information formula with a penalty term to calculate an information amount of features in the preliminary screened feature set, ranking all the features in a descending order according to the amount of the information, and assigning a node priority degree according to ranking.

Further, a maximum number of the parent nodes of each node of the K2 algorithm is optimized, specifically: calculating mutual information and average mutual information of each feature and all other features in the preliminary screened feature set, and marking the number of times when a mutual information value of each feature and other features is greater than an average mutual information value as the maximum number of the parent nodes of the node corresponding to the feature.

Further, for a node Xᵢ in the Bayesian network, the parent node set Π_{X}_{ᵢ} is an empty set during initialization, a network score Score_{old}=g(Xᵢ, Π_{X}_{ᵢ}) is calculated, where g is a scoring function, and then a cycle of searching for the parent node of the node Xᵢ is performed; and in the cycle, when the number of the node in the set Π_{X}_{ᵢ} is less than the maximum number of the parent nodes, a node having a node priority before Xᵢ and not within Π_{X}_{ᵢ} is used as a candidate node; a node z with a largest network score g(Xᵢ, Π_{X}_{ᵢ}∩{z}) is selected in the candidate node, and a network score thereof is marked as Score_{new}; if Score_{new}>Score_{old}, a value of the Score_{new} is assigned to Score_{old}, Πxᵢ=Π_{X}_{ᵢ}∩{z} is set, and a next round of iteration is performed; and the cycle is not stopped until Score_{new}≤Score_{old}, so as to obtain the parent node set of the node Xᵢ.

Further, calculation formula for the scoring function g(Xᵢ, Π_{X}_{ᵢ}) is as follows:

$$g ( {X}_{i} , {\prod }_{{X}_{i}} ) = \{ \begin{matrix}{\sum }_{i = 1}^{{n}^{″}} {\sum }_{k = 1}^{{r}_{i}} {N}_{i k} log ( \frac{{N}_{i k}}{{n}^{″}} ) , & if {\prod }_{{X}_{i}} is an empty set \\ {{\sum }^{{n}^{″}}}_{i = 1} γ {{\sum }^{\vert {D}_{{\prod }_{{X}_{i}}} \vert}}_{j = 1} {{\sum }^{{r}_{i}}}_{k = 1} {N}_{ijk} log ( \frac{{N}_{ijk}}{{N}_{ij}} ) - & if {\prod }_{{X}_{i}} is not an empty set \\ {{\sum }^{{n}^{″}}}_{i = 1} ( {r}_{i} - 1 ) \vert {{D}_{Π}}_{{x}_{i}} \vert * {n}^{″} , & \end{matrix}$$

where n″ is the number of the node in the set {Xᵢ, Π_{X}_{ᵢ}}, rᵢ is the number of all possible values of the Xᵢ, and |D_{Πx}_{ᵢ}| is the number of possible values of all nodes in Π_{X}_{ᵢ}; Nᵢₖ represents the number of data instances where the node Xᵢ takes a k^{th} value xᵢₖ in the enrolled patient dataset D; Nᵢⱼₖ represents the number of data instances where the node Xᵢ takes the k^{th} value xᵢₖ and a feature of Π_{X}_{ᵢ} takes a j^{th} value in the enrolled patient dataset D, and Nᵢⱼ is the number of data instances where the feature of Π_{X}_{ᵢ} takes the j^{th} value; and γ is an intensity of a temporal causal effect.

Further, by considering the occurrence of the index event as the intervention and the occurrence of the reference event as the outcome, and considering confounding factors, propensity score matching method can be employed to control for the enrolled populations in the intervention group and the control group. By comparing the occurrence of outcome events between the two groups, if the average increase in adverse reactions is greater than zero, it indicates a causal relationship between the current intervention and the outcome. In other words, the selected drug is likely to induce adverse reactions.

According to a second aspect of the present application, provided is a system for discovering adverse drug reaction signals based on causal discovery; the system includes: a data acquisition module configured to collect and clean real-world electronic medical record data; an adverse drug reaction discovery module configured to discover an adverse drug reaction signal having causality; and a signal result display module configured to present a signal discovery result; the adverse drug reaction discovery module utilizing the method for discovering adverse drug reaction signals based on causal discovery to construct a patient cohort, construct a Bayesian network containing a causal property, generate a set of confounding factors, construct an intervention group and a control group based on the set of the confounding factors, evaluate a difference in an occurrence of an adverse reaction between the intervention group and the control group, and generate the adverse drug reaction signal having the causality.

The present application has the beneficial effects that the method of constructing a set of confounding factors based on Bayesian network provided by the present application starts from the data, without artificial access and prior knowledge, and retains the confounding factors in the real world to the greatest extent. Based on these confounding factors, the control group and the intervention group in the observational study are constructed, and the relationship between drugs and adverse reactions obtained from this can be considered to have causal effect, which is more valuable in clinical guidance.

### BRIEF DESCRIPTION OF DRAWINGS

FIG. 1 is a flowchart of a method for discovering adverse drug reaction signals based on causal discovery provided by an exemplary embodiment;

FIG. 2 is a schematic diagram of a Bayesian network structure including three-dimensional features provided by an exemplary embodiment;

FIG. 3 is a flow chart of Bayesian network learning provided by an exemplary embodiment; and

FIG. 4 is a structural diagram of a system for discovering adverse drug reaction signals based on causal discovery provided by an exemplary embodiment.

### DESCRIPTION OF EMBODIMENTS

In order to make the above objects, features and advantages of the present application more obvious and easy to understand, the specific embodiments of the present application will be described in detail with reference to the accompanying drawings.

In the following description, specific details are set forth in order to fully understand the present application, but the present application can also be implemented in other ways different from those described here, and those skilled in the art can make similar promotion without violating the connotation of the present application, so the present application is not limited by the specific embodiments disclosed below.

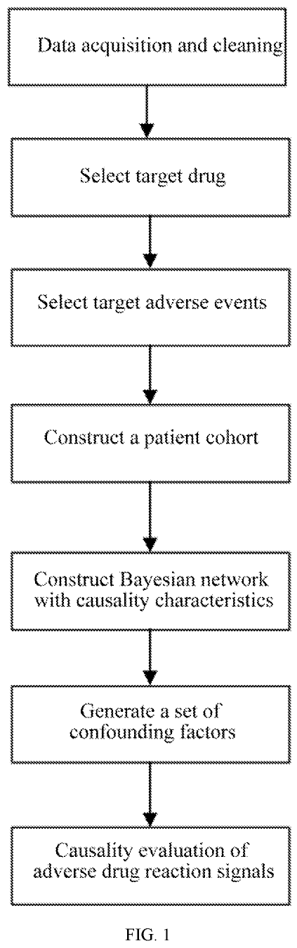

As shown in FIG. 1, an embodiment of the present application provides a method for discovering adverse drug reaction signals based on causal discovery; the method includes the following steps.

Step 1: Data Acquisition and Cleaning

Real-world patient data, medication data, diagnosis data, operation data, laboratory reports and the like are obtained from electronic medical record data, and the original date and time are retained without processing. Specifically, the obtained information includes: i) demographic information: gender, age and nationality; ii) basic medical information: allergy history, family history and blood type; iii) diagnosis and treatment information: diagnosis records, laboratory reports, medication records and operation records.

First of all, the data codes are unified: gender, age, nationality, allergic history, blood type, laboratory reports and medication information use self-designed codes, and the coding form is not limited. Diagnosis and family history use ICD-10 codes, and surgical information uses ICD-9-CM codes.

After data standardization, the data are regularly merged and transformed: gender, nationality, allergy history and blood type data are filled as classified variable data according to natural conditions; diagnosis-related features and surgical information are filled as binary variables according to the codes, that is, 1 is recorded for occurrence, otherwise 0; according to the actual situation, the laboratory reports are filled as multi-classification variables, that is, those exceeding the upper limit of the normal value of the corresponding indicators are marked as “high”, those below the lower limit of the normal value are marked as “low” and those within the normal value range are marked as “normal”; the age data are divided into four groups, namely “less than 18 years old”, “18 to 44 years old”, “45 to 59 years old” and “over 60 years old”; in the case of missing data, the whole sample is excluded if the data of gender, nationality, age and blood type is missing; the absence of diagnosis-related data and operation information is regarded as not occurring, and recorded as 0; the missing data of the laboratory reports is regarded as normal.

To sum up, the collected electronic medical record data will be cleaned and transformed into a form that can be used for the discovery of adverse drug reactions in the future.

Step 2: Construction of a Patient Cohort

First, the target drug and adverse event to be analyzed are selected. For example, the selected target drug is voriconazole and the adverse event is hepatotoxicity.

The target drug can be a single drug or a type of drugs with the same efficacy or property. When a type of drugs is selected as the target drug, the selected drugs are regarded as the same drug.

Adverse event can be defined by using diagnosis or a specific type of laboratory reports or by using both diagnosis and a specific type of laboratory reports. For example, the definition of “hepatotoxicity” can be defined according to clinical practice or clinical guidelines, using the diagnosis of “drug-induced liver injury” or the following compound rules composed of diagnosis and laboratory reports:

Alanine aminotransferase≥5×upper limit of normal value (ULN).

Alanine aminotransferase≥3×ULN with total bilirubin>2×ULN.

Alkaline phosphatase≥2×ULN, without osteopathy and elevated glutamyl transpeptidase.

If one of the above rules is met, it can be considered that the target adverse event has occurred.

In that present application, the first use of the target drug and the first occurrence of the target adverse event after the first use of the target drug are define as main event occurrence nodes, the date of the first use of the target drug is recorded as an index date, and the use of the target drug is recorded as an index event; the first occurrence of the target adverse event is recorded as a marker event, and the corresponding date is recorded as a marked date. The patient population with index events or marker events is defined as the enrolled population, and on this basis, a series of specific inclusion and a series of specific inclusion and exclusion criteria can be further defined to further screen the enrolled population, or not. The screened enrolled population constitutes a patient cohort, and the patient data in the patient cohort is recorded as an enrolled patient data set.

Step 3: Discovering Adverse Drug Reaction Signals Based on Causal Discovery.

3.1 Construction of a Set of Confounding Factors Based on a Bayesian Network

The enrolled patient data set is defined as D=<Va, T>, which contains n features {X₁, X₂, . . . , Xₙ₋₂, X_{index}, Xₘₐᵣₖₑᵣ}, in which X_{index} is a feature indicating whether the index event occurs, Xₘₐᵣₖₑᵣ is a feature indicating whether the marker event occurs, and X₁, X₂, . . . , Xₙ₋₂ is other features extracted from the electronic medical record data of the enrolled patients. The value of the feature is stored in the feature set Va, and the time when the feature occurs is stored in the time set T. The steps of constructing the set of confounding factors are as follows (unless otherwise specified, the values of the feature X in the following steps are all taken from Va):
1) Preliminary screening of feature correlation. X₁, X₂, . . . , Xₙ₋₂ is subjected to single factor logistic regression with X_{index} and Xₘₐᵣₖₑᵣ, respectively, and the features corresponding to X_{index} and Xₘₐᵣₖₑᵣ whose significance levels p are both greater than a set threshold p* are eliminated. The retained features are all features that will affect the occurrence of index events or marker events. The new feature set includes n′ features, which is recorded as a post-preliminary-screening feature set S={X₁, X₂, . . . , X_{n′-2}, X_{index}, Xₘₐᵣₖₑᵣ}.
2) Calculation of feature information. The information amount in the n′ features in post-preliminary-screening feature set is calculated by using the formula of mutual information with a penalty term, which emphasizes the relationship between {X₁, X₂, . . . , X_{n′-2}} and X_{index}, Xₘₐᵣₖₑᵣ and weakens the mutual relationship between features in {X₁, X₂, . . . , X_{n′-2}}. Assuming that S′ is a set of remaining features after feature Xᵢ is removed from the set X₁, X₂, . . . , X_{n′-2}, then the information amount I(Xᵢ) of the feature Xᵢ(i=1, 2, . . . , n′−2) is calculated as follows:

$$I ( {X}_{i} ) = {\sum }_{{X}_{i} {X}_{i n d e x}} p ( {X}_{i} , {X}_{i n d e x} ) log \frac{p ( {X}_{i} , {X}_{i n d e x} )}{p ( {X}_{i} ) p ( {X}_{i n d e x} )} + {\sum }_{{X}_{i} {X}_{m a r k e r}} p ( {X}_{i} , {X}_{mark e r} ) log \frac{p ( {X}_{i} , {X}_{mark e r} )}{p ( {X}_{i} ) p ( {X}_{mark e r} )} - α {\sum }_{{X}_{j} ∈ {S}^{′}} {\sum }_{{X}_{i} {X}_{j}} p ( {X}_{i} , {X}_{j} ) log \frac{p ( {X}_{i} , {X}_{j} )}{p ( {X}_{i} ) p ( {X}_{j} )}$$

where α is a weight factor, which may be generally determined by the scale of the number of features contained in the post-preliminary-screening feature set, and

$$α = 1 - \frac{1}{\vert s \vert}$$

can be taken. For X_{index} and Xₘₐᵣₖₑᵣ, the information amount thereof is 1. Therefore, the calculation formula of the corresponding information amount is as follows:

$$I ( {X}_{i n d e x} ) = 1 + {\sum }_{{X}_{index} , {X}_{m a r k e r}} p ( {X}_{i n dex} , {X}_{m a r k e r} ) log \frac{p ( {X}_{i n dex} , {X}_{mark e r} )}{p ( {X}_{i n d e x} ) p ( {X}_{mark e r} )} - α {\sum }_{{X}_{j} ∈ {S}^{′}} {\sum }_{{X}_{index} , {X}_{j}} p ( {X}_{i n dex} , {X}_{j} ) log \frac{p ( {X}_{i n dex} , {X}_{\dot{j}} )}{p ( {X}_{i n d e x} ) p ( {X}_{j} )} I ( {X}_{m a r k e r} ) = {\sum }_{{X}_{m a r k e r} , {X}_{i n d e x}} p ( {X}_{mark e r} , {X}_{i n d e x} ) log \frac{p ( {X}_{marker} , {X}_{i n d e x} )}{p ( {X}_{marker} ) p ( {X}_{i n d e x} )} + 1 - α {\sum }_{{X}_{j} ∈ {S}^{′}} {\sum }_{{X}_{marker} , {X}_{j}} p ( {X}_{marker} , {X}_{j} ) log \frac{p ( {X}_{marker} , {X}_{j} )}{p ( {X}_{marker} ) p ( {X}_{j} )}$$

3) Bayesian network structure learning. According to the method, causal features are introduced into the process of screening confounding factors, and the traditional K2 algorithm is improved to learn a Bayesian network structure from the grouped patient data set, so that the relationship among the features in the data set can be expressed as accurately as possible. K2 algorithm is a Bayesian network structure learning algorithm based on scoring. In order to reduce the search space, it is necessary to provide the algorithm with a priori priority of nodes and the maximum number of parent nodes of each node. According to the features of the data set of the enrolled patients, an improvement on the determination process of the above two key parameters are proposed, which is specifically as follows.

First, the optimized node priority is calculated. All the features are sorted in a descending order according to the feature information amount in the previous step, and the first feature is assigned with a node priority of 1, the second feature is assigned with a node priority of 2, and so on. If the information of multiple features is equal, they are recorded as juxtaposition, and they are assigned with the same node priority. If the priorities of m nodes are the same, the sum of mutual information between these features and X_{index} and Xₘₐᵣₖₑᵣ are calculated respectively, that is:

$${I}^{′} ( {X}_{i} ) = {\sum }_{{X}_{i ,} {X}_{index}} p ( {X}_{i} , {X}_{index} ) log \frac{p ( {X}_{i} , {X}_{index} )}{p ( {X}_{i} ) p ( {X}_{index} )} + {\sum }_{{X}_{i} , {X}_{marker}} p ( {X}_{i} , {X}_{marker} ) log \frac{p ( {X}_{i} , {X}_{marker} )}{p ( {X}_{i} ) p ( {X}_{marker} )}$$

I′ is sorted in a descending order, the priority of the first feature node is not added with score, and the priority of the second feature node is increased by 1/m, and so on, so as to obtain the node priority ranking of each feature.

Second, the optimized maximum number of parent nodes. The method of using the same maximum number of parent nodes for each feature in the original K2 algorithm is changed. A dynamic algorithm is used in the present application. First, the mutual information MI and the average mutual information Avg_MI of each feature and all other features are calculated. The mutual information MI of the feature Xᵢ and Xᵢ (Xⱼ, XⱼϵS) is calculated as follows:

$$MI ( {X}_{i} , {X}_{j} ) = {\sum }_{{X}_{i} , {X}_{j}} p ( {X}_{i} , {X}_{j} ) log \frac{p ( {X}_{i} , {X}_{j} )}{p ( {X}_{i} ) p ( {X}_{j} )}$$

The formula for calculating the average mutual information Avg_MI of feature Xᵢ is as follows:

$$Avg\_MI ( {X}_{i} ) = \frac{1}{{n}^{′}} {\sum }_{{X}_{j} ∈ S} {\sum }_{{X}_{i} , {X}_{j}} p ( {X}_{i} , {X}_{j} ) log \frac{p ( {X}_{i} , {X}_{j} )}{p ( {X}_{i} ) p ( {X}_{j} )}$$

The number of times that the mutual information value between each feature and other features is greater than Avg_MI value is taken as the estimated value of the number of parent nodes of the node, and it is recorded as the maximum number of parent nodes of the node.

Finally, the learning of a Bayesian network structure. In the learning process of a Bayesian network structure, the present application introduces one of the essential properties of causality, that is, “cause” occurs before “effect”. Therefore, the network to be learned by the present application is a n′-dimensional Bayesian network, which is denoted as B=(X, G, Θ), where X is the n′-dimensional feature vector; G=(N, E) is a directed acyclic graph, N={X₁, X₂, . . . , X_{n′-2}, X_{index}, Xₘₐᵣₖₑᵣ} is a node of the directed acyclic graph, and E is an edge of the directed acyclic graph, which represents the dependency between features. Θ={θᵢⱼₖ}_{i=1 . . . n′,jϵD}_{_{Πxi}}_{,k=1 . . . r}_{ᵢ} is a parameter of the network, in which θᵢⱼₖ=P(Xᵢ=xᵢₖ|Π_{X}_{ᵢ}=wᵢⱼ); Π_{X}_{ᵢ} represent the set of all the parent nodes of Xᵢ in a graph G, D_{Πx}_{ᵢ} represents the possible values of all the nodes in Π_{X}_{ᵢ}, rᵢ is the number of all possible values of Xᵢ, xᵢₖ is the k^{th} value of the feature Xᵢ, wᵢⱼ is the j^{th} value of the feature Π_{X}_{ᵢ}, and θᵢⱼₖ is the probability of taking the value of xᵢₖ under the condition that all the parent nodes of the node Xᵢ take the value of wᵢⱼ.

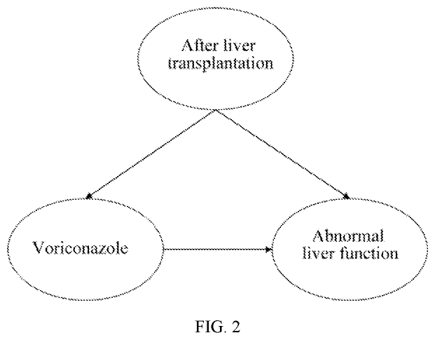

The meanings of N, Π_{X}_{ᵢ}, D_{Πx}_{ᵢ}, wᵢⱼ and xᵢₖ will be explained through an example. FIG. 2 is a schematic diagram of a Bayesian network structure, which contains three-dimensional features, namely N={after liver transplantation, voriconazole, abnormal liver function}. Let the feature Xᵢ=abnormal liver function. For the node with abnormal liver function, it has two parent nodes, “after liver transplantation” and “voriconazole”, namely, Π_{X}_{ᵢ}={after liver transplantation, voriconazole}. The possible values of the parent node include four conditions, namely, “not after liver transplantation, not taking voriconazole”, “after liver transplantation, not taking voriconazole”, “not after liver transplantation, taking voriconazole” and “after liver transplantation, taking voriconazole”, and the corresponding data can be expressed as wᵢⱼ having four values, D_{Πx}_{ᵢ}, ={{0,0}, {1,0}, {0,1}, {1,1}}, j=0,1,2,3; the “abnormal liver function” node itself has two possibilities, namely “normal liver function” and “abnormal liver function”, and the corresponding data is expressed as xᵢₖ having two values for: 0 and 1, where k=0,1.

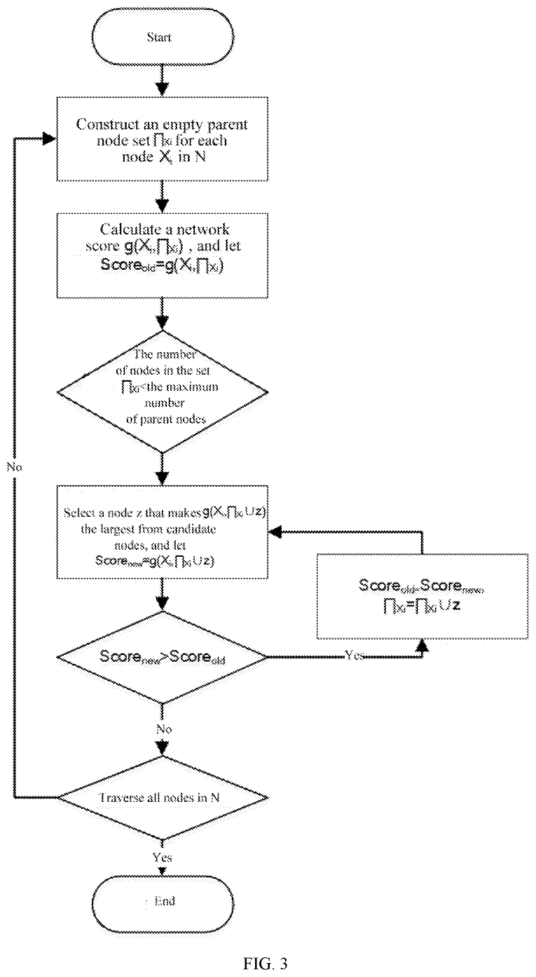

As shown in FIG. 3, for a node Xᵢ in N, its parent node set Π_{X}_{ᵢ} is set as an empty set 0 during initialization, and the network score {Π_{X}_{ᵢ}, Xᵢ} of the set Score_{old}=g(Xᵢ, Π_{X}_{ᵢ}) is calculated, and then the cycle of searching the parent nodes of the node Xᵢ is entered. In the cycle, when the number of the node in the set Π_{X}_{ᵢ} is less than the maximum number of the parent nodes, for a node z having a node priority before Xᵢ and not within Π_{X}_{ᵢ}, g(Xᵢ, Π_{X}_{ᵢ}∩{z}) is calculated; the node z with argmax_{z}(g(Xᵢ, Π_{X}_{ᵢ}∩{z}) is taken, and Score_{new}=g(Xᵢ, Π_{X}_{ᵢ}∩{z}) is compared with Score_{old}. If Score_{new}>Score_{old}, a value of the Score_{new} is assigned to Score_{old}, Π_{X}_{ᵢ}=Π_{X}_{ᵢ}∩{z} is set, and a next round of iteration is performed; and the cycle is not stopped until Score_{new}≤Score_{old}, so as to obtain the parent node set of the node Xᵢ.

In the above calculation process, the scoring function g(Xᵢ, Π_{X}_{ᵢ}) is scored by the improved Bayesian information standard with a penalty term. Because the maximum number of parent nodes estimated by the previous optimization of the present application may be greater than the actual number of parent nodes, this will bring redundant causality to the network, so the scoring function used in the present application is calculated according to the following formula:

$$g ( {X}_{i} , {\prod }_{{X}_{i}} ) = \{ \begin{matrix}{\sum }_{i = 1}^{{n}^{″}} {\sum }_{k = 1}^{{r}_{i}} {N}_{i k} log ( \frac{{N}_{i k}}{{n}^{″}} ) , & if {\prod }_{{X}_{i}} - ∅ \\ {\sum }_{i = 1}^{{n}^{″}} γ {\sum }_{i = 1}^{\vert {D}_{{\prod }_{{X}_{i}}} \vert} {\sum }_{k = 1}^{{r}_{i}} {N}_{ijk} log ( \frac{{N}_{ijk}}{{N}_{ij}} ) - & if {\prod }_{{X}_{i}} = ∅ \\ {\sum }_{i = 1}^{{n}^{″}} ( {r}_{i} - 1 ) \vert {{D}_{Π}}_{{x}_{i}} \vert * {n}^{″} , & \end{matrix}$$

where n″ is the number of the node in the set {Xᵢ, Π_{X}_{ᵢ}}; Nᵢₖ represents the number of data instances where the node Xᵢ takes a k^{th} value xᵢₖ in the enrolled patient dataset D; Nᵢⱼₖ represents the number of data instances where the node Xᵢ takes the k^{th} value xᵢₖ and a feature of Π_{X}_{ᵢ} takes a j^{th} value in the enrolled patient dataset D, and Nᵢⱼ=Σₖ₌₁^{r}^{ᵢ}=Nᵢⱼₖ is the number of data instances where the feature of Π_{X}_{ᵢ} takes the j^{th} value; |D_{Πx}_{ᵢ}| represents the number of possible values of all nodes in Π_{X}_{ᵢ}; γ is the strength of a temporal causal effect, and its size reflects the strength of this causal effect that “cause” occurs before “effect”; for a feature s in Π_{X}_{ᵢ}, the instance ratio of the occurrence time Tₛ<T_{X}_{ᵢ} is calculated, and when this ratio is greater than the set threshold β (β=0.5 in this embodiment), γₛ=1 is recorded, and otherwise γₛ=0. The calculation method of γ is:

$$γ = \frac{1}{\vert {D}_{{Π}_{{x}_{i}}} \vert} {\sum }_{s ∈ {D}_{{Π}_{{x}_{i}}}} γ s$$

In the calculation formula of the scoring function, the second term is a penalty term, and Σᵢ₌₁^{n″}(rᵢ−1)|D_{Πx}_{ᵢ}|*n″ represents the complexity of the network. The addition of n″ can also eliminate the over-fitting problem of the network caused by the large estimated value of the largest parent node to some extent.
4) Construction of the set of confounding factors for drug-adverse reaction signal discovery. In the Bayesian network calculated above, a common parent node of X_{index} and Xₘₐᵣₖₑᵣ is considered as a factor that affects whether the index event and the marker event occur at the same time, and is used as a set of confounding factors in the subsequent causal evaluation of adverse drug reaction signals.

3.2 Causality Evaluation of Drug-Adverse Reaction Signals Based on Propensity Score Matching

Propensity score matching is a technique often used in clinical observational studies to control confounding deviation, which is the possibility that individuals with specific features are assigned to the intervention group (relative to the control group), that is, propensity score=p(Z=1|X), where Z is intervention, all the data of the intervention group Z=1, the data of the control group Z=0, and X is a given condition. In the real-world observational study, the method of propensity score matching can make the confounding factors of the cohort sample of the intervention group and the control group well controlled, so as to achieve the purpose of simulating the randomized controlled trial and obtain the clinical conclusion with causality.

In the present application, whether the index event occurs is considered as an intervention Z and whether the flag event occurs is considered as an end Y According to the set of confounding factors constructed based on the Bayesian network, the people who enter the intervention group and the control group are controlled by the method of propensity score matching, and the results of drug-adverse reaction signals with causal effects are obtained by comparing the occurrence of end events between the two groups. The specific methods are as follows:

Firstly, an intervention group cohort Cohort_{Case} is constructed, and all patients with index events are screened into the group. According to the confounding factor set, the confounding factor data set of the intervention group is constructed by using the confounding factor data of the patients in the cohort, and the propensity score of each sample in the intervention group cohort is calculated by logistic regression.

Secondly, a control group cohort Cohort_{Control} is constructed, and all patients without index events are screened into the group. According to the confounding factor set, the confounding factor data set of the control group is constructed by using the confounding factor data of the patients in the cohort, and the propensity score of each sample in the control group is calculated by using logistic regression.

Thirdly, stratified propensity score matching based on patient similarity. The propensity score of the intervention group is sorted in descending order, and is divided into 1/μ propensity score intervals with μ(0<μ<1) as the interval. The control group is divided into several propensity scoring intervals by the same method. For the sample case in each intervention group, the sample with the smallest distance from the case itself is selected as a match in the propensity score interval corresponding to the control sample, that is, the patient sample most similar to the patient corresponding to the case sample is selected as a match, and control group samples are formed from the matched samples. Assuming that the data set of confounding factors in the intervention group/control group contains c confounding factor features, the distance d(i, j) between samples i and j adopts the following distance calculation formula:

$$d ( i , j ) = \frac{{\sum }_{f = 1}^{c} {δ}_{ij}^{( f )} {d}_{ij}^{( f )}}{{\sum }_{f = 1}^{c} {δ}_{ij}^{( f )}}$$

where if the sample i or j does not have the metric value of the f^{th} feature, the item δᵢⱼ^{(f)}=0 (the present application completes data filling in the process of data cleaning, so the above situation does not exist); and otherwise, the indicating item δᵢⱼ^{(f)}=1. dᵢⱼ^{(f)} is the contribution of the f^{th} feature to the dissimilarity between i and j. For binary classification features, there are only two states, and the two states have the same value and weight. When the corresponding binary feature values of sample i and sample j are the same, dᵢⱼ^{(f)} is set to 0; otherwise, dᵢⱼ^{(f)} is set to 1. For multi-classification features, it is a generalization of binary features, and more than two state values can be taken. Similar to binary features, the present application defines that when the feature values of the f^{th} attribute of sample i and sample j are the same, dᵢⱼ^{(f)} is set to 0; and otherwise, dᵢⱼ^{(f)} is set to 1.

Fourthly, the average gain ASG of occurrence of adverse reactions is calculated, and the calculation formula is as follows:

$$ASG = E [ Y \vert Z = 1 ] - E [ Y \vert Z = 0 ] = \frac{1}{{n}_{1}} {\sum }_{i = 1}^{{n}_{1}} {Y}_{i} - \frac{1}{{n}_{0}} {\sum }_{i = 1}^{{n}_{0}} {Y}_{i}$$

where E stands for expectation, n₀ and n₁ represent the numbers of patients in the control group and the intervention group respectively; for a patient i, Yᵢ stands for the occurrence of a marker event, and when a marker event occurs, Yᵢ=1, and otherwise Yᵢ=0. In this example, n₀=n₁, so the calculation result of ASG is the number of patients with marker events (adverse reactions) in the intervention group minus the number of patients with marker events (adverse reactions) in the control group, and then divided by the number of patients in the intervention group. When ASG>0, there is a causality between the current intervention and the outcome, that is, the currently selected drug will cause adverse reactions.

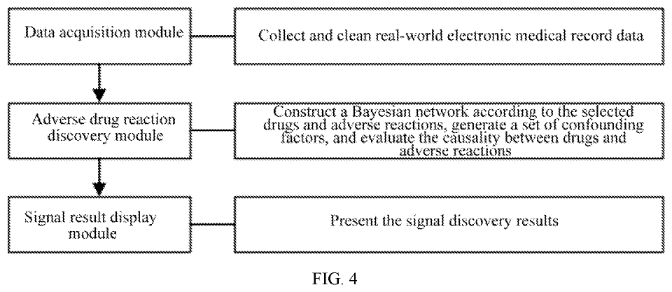

As shown in FIG. 4, the present application also provides an embodiment of an adverse drug reaction signal discovery system based on causal discovery, which includes:
a data acquisition module configured to collect and clean real-world electronic medical record data;
an adverse drug reaction discovery module configured to discover an adverse drug reaction signal having causality; and
a signal result display module configured to present a signal discovery result.

The adverse drug reaction discovery module is a core module in the present application. It utilizes the aforementioned the adverse drug reaction signal discovery method based on causal discovery. The module constructs a patient cohort, builds a Bayesian network incorporating causal characteristics, generates a set of confounding factors, creates intervention and control groups based on the confounding factors, evaluates the differences in adverse reaction occurrences between the two groups, and generates adverse drug reaction signals with causal relationships.

The present application is not limited to the existing drug-adverse reaction relationship, and the adverse drug reaction signal can be found by using the real-world electronic medical record data, so that the drug-adverse reactions that are not shown in the clinical trial stage can be identified, which is of great significance for the safe development of clinical activities.

The present application is not limited to finding the correlation between drugs and adverse reactions, and generates the most comprehensive set of confounding factors by introducing causal features into the Bayesian network construction process, and achieves the effect of simulating random controlled trials by controlling these confounding factors, so as to evaluate and verify the causality between drugs and adverse reactions.

In this application, the term “controller” and/or “module” may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components (e.g., op amp circuit integrator as part of the heat flux data module) that provide the described functionality; or a combination of some or all of the above, such as in a system-on-chip.

The term memory is a subset of the term computer-readable medium. The term computer-readable medium, as used herein, does not encompass transitory electrical or electromagnetic signals propagating through a medium (such as on a carrier wave); the term computer-readable medium may therefore be considered tangible and non-transitory. Non-limiting examples of a non-transitory, tangible computer-readable medium are nonvolatile memory circuits (such as a flash memory circuit, an erasable programmable read-only memory circuit, or a mask read-only circuit), volatile memory circuits (such as a static random access memory circuit or a dynamic random access memory circuit), magnetic storage media (such as an analog or digital magnetic tape or a hard disk drive), and optical storage media (such as a CD, a DVD, or a Blu-ray Disc).

The apparatuses and methods described in this application may be partially or fully implemented by a special purpose computer created by configuring a general-purpose computer to execute one or more particular functions embodied in computer programs. The functional blocks, flowchart components, and other elements described above serve as software specifications, which can be translated into the computer programs by the routine work of a skilled technician or programmer.

The above is only the preferred embodiment of one or more embodiments of this specification, and it is not used to limit one or more embodiments of this specification. Any modification, equivalent substitution, improvement and the like made within the spirit and principle of one or more embodiments of this specification shall be included in the scope of protection of one or more embodiments of this specification.

What is claimed is:

1. A method for discovering adverse drug reaction signals based on causal discovery, comprising the following steps:
acquiring and cleaning real-world electronic medical record data;
selecting a target drug and an adverse event, marking use of the target drug as an index event and an appearance of a target adverse event as a marker event, and constructing a patient cohort according to a patient population in which the index event or the marker event occurs; and
generating a set of confounding factors affecting both a medication intervention and an occurrence of an adverse reaction by constructing a Bayesian network containing a causal property, wherein said generating the set of the confounding factors comprises:
marking patient data in the patient cohort as an enrolled patient dataset, wherein the enrolled patient dataset comprises a feature X_{index} indicating whether the index event occurs, a feature Xₘₐᵣₖₑᵣ indicating whether the marker event occurs, and other features of an enrolled patient extracted from the electronic medical record data;
forming a preliminary screened feature set by retaining features capable of affecting occurrence of the index event or the marker event through a single-factor logistic regression method;
taking feature in the preliminary screened feature set as a node of the Bayesian network, learning a Bayesian network structure from the enrolled patient dataset according to a K2 algorithm, introducing a causality in a learning process of the Bayesian network structure, obtaining a parent node set of a node after a plurality of rounds of iterations, taking a common parent node of the features X_{index} and Xₘₐᵣₖₑᵣ as factors affecting whether both the index event and the marker event occur, and generating the set of the confounding factors;
optimizing a node priority of the K2 algorithm, comprising: calculating an information amount of features in the preliminary screened feature set using a mutual information formula with a penalty term, ranking all the features in a descending order according to the amount of the information, and assigning a node priority degree according to ranking;
optimizing a maximum number of parent nodes of each node of the K2 algorithm, comprising: calculating mutual information and average mutual information of a feature and all other features in the preliminary screened feature set, and marking a number of times when a mutual information value of the feature and other features is greater than an average mutual information value as the maximum number of the parent nodes of a node corresponding to the feature; and
constructing cohorts of an intervention group and a control group based on the set of the confounding factors, simulating a randomized controlled trial, evaluating a difference in occurrences of adverse reactions between the intervention group and the control group, and generating an adverse drug reaction signal having the causality.

2. The method for discovering adverse drug reaction signals based on causal discovery according to claim 1, wherein the target drug is a single drug, a type of drugs having a same efficacy, or a type of drugs having a same property; and
the adverse event is defined by a diagnosis, a specific type of laboratory reports, or both the diagnosis and the specific type of laboratory reports.

3. The method for discovering adverse drug reaction signals based on causal discovery according to claim 1, wherein the patient population in which the index event or the marker event occurs is defined as an enrolled population, inclusion and exclusion criteria is defined to screen the enrolled population, the screened enrolled population constitutes the patient cohort, and the patient data in the patient cohort constitutes the enrolled patient dataset.

4. The method for discovering adverse drug reaction signals based on causal discovery according to claim 1, wherein a parent node set Π_{X}_{ᵢ} of a node Xᵢ in the Bayesian network is an empty set when the node Xᵢ is initialized, a network score Score_{old}=g(Xᵢ, Π_{X}_{ᵢ}) is calculated, where g represents a scoring function, and a cycle of searching for a parent node of the node Xᵢ is performed; and wherein in the cycle, when a number of nodes in the set Π_{X}_{ᵢ} is less than a maximum number of the parent nodes, a node having a node priority before the node Xᵢ and not within the set Π_{X}_{ᵢ} is used as a candidate node; a node z with a largest network score g(Xᵢ, Π_{X}_{ᵢ} ∩{z}) is selected in the candidate node, and a network score of the node z is denoted as Score_{new}; when Score_{new}>Score_{old}, a value of Score_{new} is assigned to Score_{old}, Π_{X}_{ᵢ}=Π_{X}_{ᵢ}∩{z} is set, and a next round of iteration is performed; and the cycle stops until Score_{new}≤Score_{old}, so as to obtain the parent node set of the node Xᵢ.

5. The method for discovering adverse drug reaction signals based on causal discovery according to claim 4, wherein a scoring function g(Xᵢ, Π_{X}_{ᵢ}) is calculated as follows
$$g ( {X}_{i} , {\prod }_{{X}_{i}} ) = \{ \begin{matrix}{\sum }_{i = 1}^{{n}^{″}} {\sum }_{k = 1}^{{r}_{i}} {N}_{i k} log ( \frac{{N}_{i k}}{{n}^{″}} ) , & if {\prod }_{{X}_{i}} = ∅ \\ {{\sum }^{{n}^{″}}}_{i = 1} γ {{\sum }_{j = 1}}^{\vert {D}_{{\prod }_{{X}_{i}}} \vert} {{\sum }^{{r}_{i}}}_{k = 1} {N}_{ijk} log ( \frac{{N}_{ijk}}{{N}_{ij}} ) - & if {\prod }_{{X}_{i}} \neq ∅ \\ {{\sum }^{{n}^{″}}}_{i = 1} ( {r}_{i} - 1 ) \vert {{D}_{Π}}_{{x}_{i}} \vert * {n}^{″} , & \end{matrix}$$
where n″ represents a number of nodes in the set {Xᵢ, Π_{X}_{ᵢ}}, rᵢ represents a number of all possible values of the node Xᵢ, and |D_{Πx}_{ᵢ}| represents a number of possible values of all nodes in the set Π_{X}_{ᵢ}; Nᵢₖ represents a number of data instances where the node Xᵢ takes a k^{th} value xᵢₖ in an enrolled patient dataset D; Nᵢⱼₖ represents a number of data instances where the node Xᵢ takes the k^{th} value xᵢₖ and a feature of the set Π_{X}_{ᵢ} takes a j^{th} value in the enrolled patient dataset D, and Nᵢⱼ represents a number of data instances where the feature of the set Π_{X}_{ᵢ} takes the j^{th} value; and γ is an intensity of a temporal causal effect.

6. The method for discovering adverse drug reaction signals based on causal discovery according to claim 1, comprising: by considering the occurrence of the index event as the intervention and the occurrence of the reference event as the outcome, and considering confounding factors, propensity score matching method can be employed to control for the enrolled populations in the intervention group and the control group. By comparing the occurrence of outcome events between the two groups, if the average increase in adverse reactions is greater than zero, it indicates a causal relationship between the current intervention and the outcome. In other words, the selected drug is likely to induce adverse reactions.

7. A system for discovering adverse drug reaction signals based on causal discovery, comprising: a data acquisition module configured to collect and clean real-world electronic medical record data; an adverse drug reaction discovery module configured to discover an adverse drug reaction signal having the causality; and a signal result display module configured to present a signal discovery result; wherein the adverse drug reaction discovery module constructs a patient cohort with the method according to claim 1, constructs a Bayesian network containing a causal property, generates a set of confounding factors, constructs an intervention group and a control group based on the set of the confounding factors, evaluates a difference in an occurrence of an adverse reaction between the intervention group and the control group, and generates the adverse drug reaction signal having the causality.
